(19) United States
(12) Patent Application Publication
(10) Pub. No.: US 2025/0221791 A1
(43) Pub. Date: Jul. 10, 2025

(54) HOLDING SYSTEM FOR AN OPERATOR

(71) Applicant: HELLSTERN MEDICAL GMBH, Wannweil (DE)

(72) Inventors: Sabrina HELLSTERN, Wannweil (DE); Claudia SODHA, Gechingen (DE); Alexander STROBEL, Haigerloch-Stetten (DE); Harald RAGER, Bisingen-Wessingen (DE)

(21) Appl. No.: 18/837,737

(22) PCT Filed: Feb. 9, 2023
(86) PCT No.: PCT/EP2023/053180

§ 371 Date: Aug. 12, 2024

(30) Foreign Application Priority Data
Feb. 10, 2022 (DE) 10 2022 103 175.6

(51) Int. Cl. A61B 90/60

(52) U.S. Cl. CPC A61B 90/60

(57) ABSTRACT

The holding system according to the invention has a support device including a plurality of adjustment axes and an operating device. The support device is configured to hold at least one part of the operator's body received by the support device in a position and/or orientation. The operating device is configured to control the adjustment axes and has at least one first operating unit arranged outside the operator's gripping range and a second operating unit arranged in the operator's gripping range, with at least partially corresponding functionality. Preferably, the operating device also has a third operating unit with which the operator can control the support device with their body movements, as well as a fourth operating unit arranged in the operator's gripping range. This divides the functionality of the support device between the control units, thus achieving intuitive control.

### CROSS REFERENCE TO RELATED APPLICATIONS

This application is a National Stage of PCT Application No. PCT/EP2023/053180 filed on Feb. 9, 2023, which claims priority to German Patent Application No. DE 10 2022 103 175.6 filed on Feb. 10, 2022, the contents each of which are incorporated herein by reference thereto.

### TECHNICAL FIELD

The present disclosure refers to a holding system for an operator in the surgery as well as for laboratory personnel or dentists.

### BACKGROUND

Holding systems for operators are in general known. For example, operating chairs having arm and/or foot rests for the operator exist that allow that the operator usually standing at the operating table is held by the operating chair or by the arm and/or foot rests. Thereby, particularly during long lasting operations in specific forced postures and/or operations with particular requirements to the fine motor skills of the operator, the operator can carry them out in a manner with low or without fatigue. In the ideal case, the operator can carry out such operations in reliable and high quality.

Operating chairs are usually adjustably configured in order to be adaptable to the requirements of the operator during the operation. For example, an operating chair is known from DE 10 2020 103 861 B3 that can be adjusted in the height as well as in a lateral direction and in forward-backward-direction.

DE 20 2018 102 964 U1 describes an operating chair adjustable in height having two foot rests, wherein an adjustment of the height of the operating chair can be carried out by the operator sitting on the operating chair by means of foot pedals. The operating chair can in addition store the adjusted height and can move to the stored height in case of restart.

Moreover, an operating chair having a foot button and an upper body belt is described in U.S. Pat. No. 6,224,154 B1. The height of the seat and the foot rest as well as the forward-backward-inclination of the seat and the foot rest can be adjusted by the operator himself by means of an operating device. The operating chair illustrated there comprises in addition a main column on which an anchoring for the harness is attached, so that the operator can lean forward into the harness, wherein his upper body is supported in an adjusted position.

In view of the increasing number of functions and adjustment possibilities of operating chairs, a manual adjustment is thereby obvious, that is an adjustment by means of an operating device, which can be manually operated by the operator, because an adjustment by foot can result in incorrect adjustments of the operating chair in case of a high number of operating elements.

From the field of computer or computer-related environments, user interfaces for manual input of control signals are known, for example from DE 60 2004 006 227 T2, which particularly comprise a high functionality with a plurality of input sensitive axes, along which a control signal is detected by the user interface. The operation of such user interfaces, particularly without incorrect adjustment, however requires a high degree of concentration of the user during the input. This can distract the operator from the actual operations.

### BRIEF SUMMARY

Starting therefrom it is the object of the present disclosure to provide a holding system for an operator having an improved, as simple and as intuitive as possible operability.

This object is solved by means of the holding system for an operator, including: a support device comprising multiple adjustment axes which are configured to hold at least one body part of the operator supported by the support device in a position and/or orientation; an operating device for control of the adjustment axes, which includes at least a first operating unit arranged outside of a gripping range of the operator and a second operating unit arranged inside the gripping range of the operator, having at least partly coinciding functionality.

The holding system according to the present disclosure includes a support device comprising multiple adjustment axes and an operating device. The support device is configured to hold at least one body part of the operator supported by the support device in a position and/or orientation. The operating device is configured for control of the adjustment axes and includes at least one first operating unit arranged outside of the gripping range of the operator and a second operating unit arranged inside the gripping range of the operator having at least partly coinciding functionality.

In doing so, it becomes possible to separate the control of the adjustment axes on multiple operating units. The first operating unit is arranged outside the gripping range of the operator, so that, for example, another person, such as an assistant, can support the operator for controlling the adjustment axes of the support device. This reduces the risk of an incorrect control of the adjustment axes by the operator, who can concentrate on the operation without distraction as far as possible. For example, the operator can in this manner control or readjust a desired adjustment axis of the support device, in that he describes the additional person in which position and/or orientation he likes to be held or what kind of change of his present position and/or orientation shall be carried out. The assistant can then adjust the desired position and/or orientation by means of the second operating unit.

A particularity of the holding system according to the present disclosure is the operating device of the support device by means of which the position and/or orientation can be adjusted. The operating device includes preferably additionally a third and a fourth operating unit. The second, third and fourth operating unit are particularly arranged within the gripping range and/or foot range of the operator in distributed manner, so that the operator can control the adjustment axes of the support device during the operation. Each of the second, third and fourth operating units are assigned to specific adjustment axes of the multiple adjustment axes of the support device. A functionality defined by all of the adjustment axes of the support device is in this manner distributed on the second, third and fourth operating unit. This allows a control of the adjustment axes that is particularly intuitive for the operator, because each functionality is assigned a specific location, for example gripping range of the right or left arm of the operator and/or foot range of the right or left foot of the operator. With the first operating unit arranged outside of the gripping range as well as outside the foot range of the operator, particularly all of the adjustment axes can be controlled.

The gripping range describes a range of the support device, which the operator can reach with his right or left hand. The foot range describes a range that the operator can reach with his right or left foot. For example, a support device can be an operating chair, which is configured so that the operator can sit down thereon during the operation. The position and/or orientation of the operator sitting on the operating chair is supported by the operating chair. A functionality of an operating unit preferably describes a movement for adjustment of a position and/or orientation of the support device controlled by one or more of the adjustment axes. An adjustment axis is particularly a device that is configured to move at least a part of the support device in an adjustment direction. The part of the support device can be, for example, a lateral support or a seat of an operating chair. The movement of the adjustment axes can be translational and/or rotational. For example, the operating chair can comprise a column support, which can be, by means of the adjustment device, exclusively adjustable in height and pivotably configured in lateral direction. On the column support, additional support elements can be attached, such as an upper body retaining device, at least one, preferably multiple lateral supports and/or arm rests, as well as the seat, for example, via a support arm attached to the column support. The additional support elements are preferably arranged in an upper end section of the column support.

Particularly, the adjustment axes that can be controlled by means of the second control unit can also be controlled by the first control unit, whereby an assistant controlling the first control unit can also control the adjustment axes that the operator can control by means of the second control unit.

In an embodiment the operating device includes a third operating unit having a release device that is preferably arranged in the foot range of the operator. The release device is particularly configured to switch at least one of the adjustment axes of the support device from a holding mode into a movement mode and vice versa, wherein the supported body part of the operator can be moved in the movement mode and is held in the holding mode by means of the support device. The operator can switch the support device from the holding mode into the movement mode and can in this manner move the supported body part in an adjustment direction. For example, the predetermined time can be in a range between 0.5 and 4 s, preferably between 1 and 3 s, preferably between 2 and 3 s or also minimum 5 s. The predetermined time during which the supported body part of the operator has to rest in a constant position, so that the control device switches from the movement mode into the holding mode, can be adjusted to a duration which is found to be comfortable for the operator. The supported body part of the operator particularly takes an at least substantially constant position if the movement speed of the body part in the adjustment direction is approximately 0 m/s, that is within a tolerance range around the value 0 m/s. The limit values of the tolerance range can be, for example, ±0.2 m/s, ±0.1 m/s or ±0.05 m/s. In addition, the limit values of the tolerance range as well as the duration can be adjusted, so that it is found to be comfortable by the operator.

Preferably, the third operating unit includes a sensor device having a plurality of sensor units for detecting of movements of the operator in adjustment direction of the adjustment axes and/or the support device in adjustment direction of at least one of the adjustment axes. Particularly, the sensor signals of the sensor unit serve as control signal of the third operating unit.

It is preferred that the operating device includes a control device, which is configured to switch the support device from the movement mode into the holding mode if the supported body part of the operator rests in a position for a predetermined time.

The operator can take a desired position and/or orientation in the movement mode that is set after termination of a short time during which the operator rests in this position. The simple adjustment of the position of the operator, which is held in the holding mode by the support device, does not require time-consuming repositionings of the support device.

Preferably, the adjustment axes that can be controlled by the third operating unit can also be controlled by the first operating unit. This allows a certain redundancy in the operating concept. It is thus possible that the operator and also the assistant can control the same adjustment axes.

Particularly, the first operating unit includes the following: a control element, which is elastically supported in a rest position and can be manually moved along at least three control axes, as well as a plurality of operating elements that, when being operated, set different assignments for the control element between control axes of the control element and adjustment axes. In addition, the control element can be movable along minimum four, preferably minimum five, particularly preferably along minimum six control axes. The control axes can comprise, for example, three axes arranged orthogonal to each other, wherein the control element can be deflected, for example, in translational and/or rotational manner out of the rest position. Thus, the control element can have minimum six degrees of freedom.

It is preferred that the first operating unit is configured to detect an amount and a direction of a manually carried out deflection of the control element out of the rest position, as well as to assign a control signal to one of the adjustment axes of the support device depending on the amount and/or the direction of the deflection. For example, the control element includes multiple direction areas, for example, minimum four, that are assigned to at least one adjustment axis. In doing so, multiple adjustment axes can be controlled by the control element of the first operating unit.

Particularly, the second operating unit includes the following: a control element, which is elastically supported in a rest position and which can be moved manually along at least three control axes, as well as a plurality of operating elements, which define different assignments between control axes of the control element and adjustment axes for the control element in case of operation.

The second control unit is preferably configured to detect an amount and a direction of a deflection of the control element out of the rest position carried out manually, as well as to assign a control signal to one of the adjustment axes of the support device depending on the amount and/or the direction of the deflection. Also, the control element of the second operating unit can have multiple direction areas, for example four, that are assigned to minimum one adjustment axis. In doing so, multiple adjustment axes can be controlled by the control element of the second operating unit.

Preferably, the control unit is configured in case of at least substantially concurrent input at the first and second operating unit to control the adjustment axis based on the input of the second operating unit and to dismiss the input of the first operating unit, whereby the control commands of the operator that he carries out by means of the second operating unit are prioritized compared to the control commands of the assistant.

It is preferred that the control device is configured to control the adjustment axes based on the inputs of the third operating unit and to dismiss inputs at the first operating unit if the third operating unit is switched in the movement mode, whereby the control commands of the operator that he carries out by means of the third operating unit are prioritized compared to the control commands of the assistant.

Particularly, the second operating unit is located in a cavity of lateral support of the support device provided for the operator. The second operating unit can be arranged in the gripping range of the operator, wherein the risk of an unintended operation of the control element of the second operating unit, for example by the elbow of the operator during operation, is reduced. The lateral support can in addition comprise a preferably foldable forearm rest arranged at a distal end of the armrest. The forearm rest offers a rigid support position, which provides relief of his arm to the operator. In doing so, also the risk of the unintended operation of the control element of the second operating unit can be further reduced.

The first operating unit and the second operating unit comprise minimum one additional operating element that, if actuated, deactivates the respective control element of the operating unit. If the operator does not desire to carry out a control, the control element of the first as well as the second operating unit can be transferred into a deactivated condition in this manner. In this condition the risk of an unintended control by the operator and/or the assistant is further reduced.

### BRIEF DESCRIPTION OF THE DRAWINGS

Additional details of advantageous embodiments or details of the present disclosure are derived from the drawings, the description as well as the dependent claims. The drawings show:

FIG. 1 a schematic, three-dimensional view of an embodiment of the holding system according to the present disclosure,

FIG. 2 a schematic, three-dimensional view of the first operating unit,

FIG. 3 a schematic, three-dimensional view of the second operating unit, as well as

FIG. 4 a schematic, three-dimensional view of the fourth operating unit.

### DETAILED DESCRIPTION

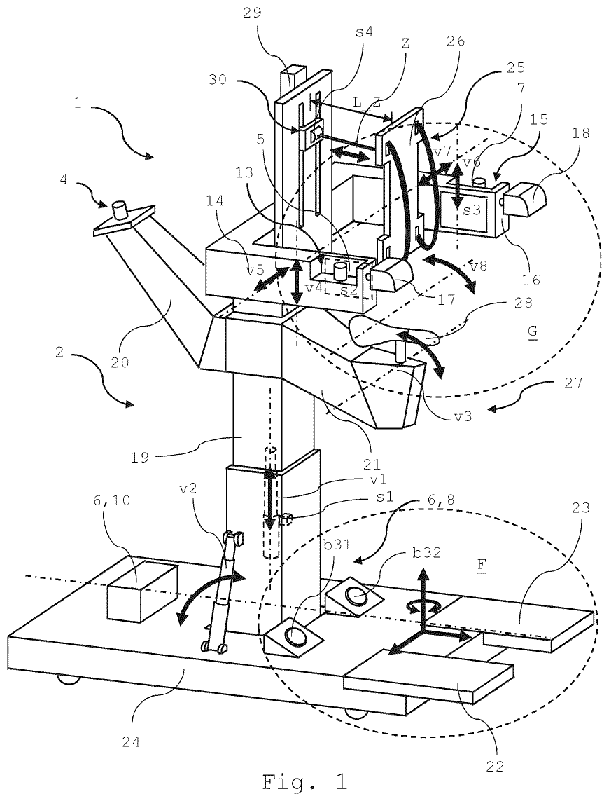

FIG. 1 illustrates the holding system 1 that serves in the illustrated example to hold an operator in a sitting position, wherein the upper body of the operator is held by a harness wearable by the operator and having a back part 26.

The support device 2 comprises a platform 24 that can be driven in forward and/or lateral direction and/or is configured to be adjustable in height. The platform comprises a footrest 22 and another footrest 23 onto which the operator can support his feet, if he is in the support device 2. The footrests 3 can in addition serve as holder for foot pedals for control of instruments, such as surgical instruments.

From a top side of the platform an operating chair 27 extends in vertical direction on which the operator can sit down. The operating chair 27 comprises a column support 19, the height of which can be adjusted, extending in vertical direction from the platform 24. The operating chair 27 comprises an adjustment axis v1 for control of the height.

The operating chair 27 with column support 19 comprises in addition a second adjustment axis v2 that is configured to pivot the operating chair 27 laterally. In this example second adjustment axis v2 comprises two linear drives (only the one in front is illustrated) that are respectively arranged laterally next to the operating chair 27. The linear drives are attached to the operating chair 27 and to the platform 2, so that a length change of the linear drives results in a pivot movement of the operating chair. The linear drives are operated in opposite direction, so that in case of a positive length change of one linear drive, the other linear drive carries out a negative length change.

The operating chair 27 additionally comprises a first support arm 20 attached to the column support 19, which extends from the column support 19 backwards. On a distal end of the first support arm 20 the first operating unit 4 of the operating device 3 is attached outside the gripping range G of the operator, so that a person standing behind the operating chair 27 can control the adjustment axis v1, . . . , vn by means of the first operating unit 4.

In addition, originating from column support 19 a second support arm 21 extends forwards. On a distal end of the second support arm 21 a seat 28 is attached for the operator. The seat 28 is configured to be inclined in a forward-backward-direction by means of a third adjustment axis v3 located in the second support arm 21.

The first support arm 20 and the second support arm 21 branch in one vertical position from column support 19. Above this vertical position a lateral support 14 and an additional lateral support 16 are attached to the column support 19.

The lateral support 14 comprises an adjustment axis v4 by means of which the lateral support 14 can be moved relative to the seat 28. The lateral support 14 comprises in addition an adjustment axis v5 by means of which the position of the lateral support can be adjusted in transverse direction.

The additional arm rest 16 also comprises an adjustment axis v6 by means of which the vertical position of the additional lateral support above the seat 28 can be adjusted. In addition, the additional lateral support 16 comprises an adjustment axis v7 by means of which the position of the additional lateral support 16 can be adjusted in transverse direction. The adjustment axes v5 and v7 are configured to move the lateral supports 14, 16 synchronously, so that they adjust in cooperating manner a distance between lateral support 14 and additional lateral support 16. Alternatively, the positions of the lateral supports 14 and 16 can be asynchronously controllable relative to one another in transverse direction.

Moreover, lateral support 14 and additional lateral support 16 can comprise a hinge device respectively, whereby the lateral support 14, 16 is manually foldable upwardly. This serves as so-called quick or emergency exit for the operator.

The lateral support 14 comprises a cavity 13, in which the second operating unit 5 of the operating device 3 is arranged. The cavity 13 of lateral support 14 is arranged on a top side of the lateral support as well as on a side facing away from the additional lateral support 16. On a distal end of the lateral support 14 a preferably foldable or extendable forearm rest 17 is arranged on which the operator can support his forearm, if he sits down on seat 28.

On a side of lateral support 14 facing the additional lateral support 16 a sensor unit s2 is attached additionally. The sensor unit s2 is configured to measure a force pressing against the lateral support 14.

The additional lateral support 16 comprises an additional cavity 15 on a top side as well as on a side facing away from the lateral support 14. In the additional cavity 15 a fourth operating unit 7 of the operating device 3 is located.

On a side of the additional lateral support 16 facing the lateral support 14 an additional sensor unit s3 is arranged. The additional sensor unit s3 is configured to measure a force pressing against the additional lateral support 16.

Moreover, a force change can be detected by sensor unit s2 and the additional sensor unit s3, such as a decrease of the force in order to initiate an erection of the operating chair. If the operator presses with the upper body against sensor unit s2 in the lateral support 14 arranged on his right side, the operating chair pivots to the right, while the operating chair pivots to the left if the operator presses his upper body against the additional sensor unit s3 in the additional lateral support 16 arranged on his left side.

Moreover, an additional forearm rest 18, on which the operator can support his forearm during the operation, is arranged on a distal end of the additional lateral support 16.

Between the lateral supports 14 and 16 and behind the seat 28, an additional support 29 extends in vertical direction. On the additional support 29, an upper body retaining device 30 having a traction means Z is attached. The traction means Z can be mechanically coupled with a back part 26 of harness 25, so that the operator can lean forward into the harness, wherein the upper body retaining device 30 is configured to apply a traction force via traction means Z on the upper body of the operator. The upper body retaining device 30 is configured to retain the upper body of the operator.

For adjusting the inclination angle in which the upper body of the operator is held by means of the upper body retaining device 30, the latter comprises an adjustment axis v8 that is configured to adjust the length of traction means Z and thus the distance between the upper body retaining device 30 and the back part 26 of harness 25. In addition, the upper body retaining device 30 can also comprise adjustment means by means of which the position can be defined at which traction means Z exits at the upper body retaining device. By means of the adjustment means, this position can be adjusted and/or locked, for example in a stepless manner or in discrete steps. In this way, the upper body retaining device 30 can be adapted to the body height of the operator. It is preferred that the upper body retaining device 30 can be adjusted, so that the position at which the traction means Z exits from the upper body retaining device 22 is arranged behind the operator sitting on the seat at least on shoulder level of the operator or higher.

The third operating unit 6 comprises a release device 8 and control device 10 communicatively connected with the release device, control device 10 being configured to switch at least one of the adjustment axes v1 . . . , vn of the support device 2 from the holding mode H into the movement mode B or vice versa in case of an actuation of the release device 8. In the present example, the release device 8 is configured to switch the adjustment axes v1, v2 and v8 from the holding mode H into the movement mode B and vice versa.

The release device 8 comprises in this example an operating element b31 arranged in the foot range F of the operator and an additional operating element b32 arranged in the foot range F of the operator. The operating elements b31 and b32 are arranged below the seat 28 on a top side of platform 24.

The third operating unit 6 comprises in addition a sensor device 9 having multiple sensor units s1, . . . , sn. The sensor signals of the sensor device 9 serve as control signals of third operating unit 6 if the support device 2 is switched in a movement mode B by releasing the release device 8. If the supported body part of the operator rests in a position and/or orientation for a predetermined time I, control device 10 switches the support device 2 from the movement mode into the holding mode.

In this example the sensor device 9 comprises four sensor units s1, s2, s3 and s4. Moreover, the sensor device 9 can, however, comprise additional sensor units s5, . . . , sn.

The sensor unit s1 is configured to detect the height of seat 28. For example, sensor unit s1 can comprise a Hall-sensor, which is attached to a gas pressure spring for height adjustment of seat 28. The sensor unit s1 can alternatively also comprise a force sensor that detects the force pressing on seat 28. The sensor unit s1 can also be configured to detect a force change. In this manner sensor unit sl can detect loading and/or relief of seat 28, for example.

In the lateral support 14 a sensor unit s2 is located on an inner side of the lateral support 14 facing seat 28. The sensor unit s2 is arranged, so that it detects a force with which upper body of the operator sitting on the seat 28 presses laterally against the lateral support 14. Similarly, in the additional lateral support 16 an additional force sensor unit s3 is located. The sensor unit s4 is located in the upper body retaining device 30 and configured to measure a length L_Z and/or a length change of the traction means Z.

For example, the upper body retaining device 30 can comprise an electrically driven winch on or from which the traction means Z can be wound. The electrical drive of the winch for the traction means can particularly be controlled, so that it drives the winch with an approximately constant torque in wind-up direction, so that an approximately constant traction force is applied on the traction means. The electrically driven winch comprises preferably a gear having a transmission ratio, whereby the winch can be configured in a self-locking manner, for example. The sensor unit s4 of upper body retaining device 30 can be as well configured to detect a force acting on traction means Z or a torque applied at the winch.

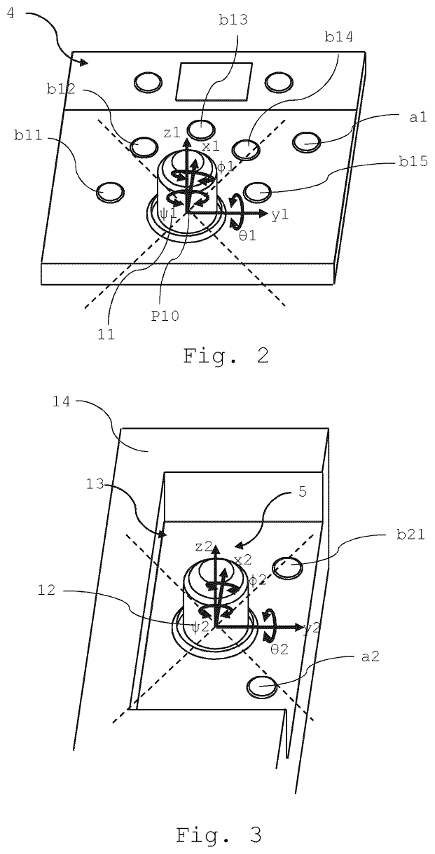

A schematic three-dimensional view of the first operating unit 4 is illustrated in FIG. 2. The first operating unit comprises a control element 11 that is elastically supported in rest position P10. The control element 11 can be moved about a first control angle ϕ1 defined by a first control axis x1, a second control angle θ1 defined by a second control axis y1 and a third control angle 41 defined by a third control axis z1. The control axes x1, y1 and z1 are arranged orthogonal to one another. The first operating unit 4 comprises in addition multiple operating elements b11, b12, b13, b14, b15 by means of which the functionality of the first operating unit 4 can be switched. The first operating unit 4 additionally comprises an additional operating element a1 that, in case of an actuation, deactivates control element 11.

FIG. 3 illustrates second operating unit 5. The second operating unit 5 is arranged in a cavity 13 of lateral support 14. The second operating unit 5 comprises a control element 12, having three control axes x2, y2 and z2 defined orthogonal to one another. A control angle ϕ2, θ2 and ψ2 is defined respectively around the control axes x2, y2, z2. The control element 12 can at least be moved in directions ϕ2, θ2 and ψ2. The second operating unit 5 comprises in addition an operating element b21 as well as an additional operating element a2 that, in case of an actuation, deactivates the control element 12 of operating unit 5.

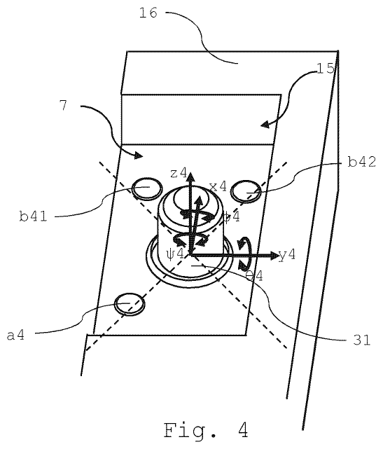

FIG. 4 illustrates the fourth operating unit 7. The fourth operating unit 7 is arranged in the additional cavity 15 of the additional lateral support 16. The fourth operating unit 7 comprises a control element 31 having three control axes x4, y4 and z4 defined orthogonal to one another. Around the control axes x4, y4, z4 respectively one control angle ϕ4, θ4 and ψ4 is defined. The control element 31 can at least be moved in the directions ϕ4, θ4 and ψ4. The fourth operating unit 7 comprises in addition to operating elements b41, b42 as well as an additional operating element a4 upon operation of which it deactivates control element 31 of fourth operating unit 7.

The holding system 1 for an operator described so far operates as follows:

For carry out an operation holding system 1 is controlled by means of operating device 3. The operator puts on the harness 25 with back part 26 and sits down on seat 28 of operating chair 27. On the back part 26 a first coupling element is attached that is complementary to a second coupling element attached to an end of traction means Z. The first coupling element and the second coupling element comprise preferably magnet elements, so that the first and second coupling element reciprocally move toward one another in a pretension position during coupling. In the pretension position the two coupling elements can be manually shifted into a latched position in which the two coupling elements releasably couple the traction means Z with back part 26 of harness 25. If the operator puts on the harness 25 first and then pulls on a preferably sterile operating gown comprising a back opening, another person can connect first coupling element with second coupling element underneath the operating gown without being required to see both coupling elements. This accelerates the procedures before the operation.

The operator can now control the height of the lateral supports 14 and 16 by means of the fourth operating unit 7 (depicted in FIG. 4), which is located in a cavity 15 of the left lateral support 16. For this purpose, the operator actuates the operating element b42. The operator can manually control the height of the lateral supports 14, 16 by pushing the control element 31 up and down in the direction of the third control axis z4. A forward or a backward shift corresponds to a deflection of the control element 31 in direction of the second control angle θ4. The operator can in addition by rotation of the control element 31 in clockwise direction and counter-clockwise direction control the distance between the lateral supports 14, 16. This corresponds to a deflection of control element 31 in direction of the first control angle ϕ4.

In addition, the operator can control the inclination of seat 28. For this purpose, the operator actuates the operating element b41 of fourth operating unit 7. The operator can now control the inclination of seat 28 by deflection of control element 31 in direction of the second control angle θ4.

If the operator is content with his adjustments, he can push the additional operating element a4, so that the control element 31 of fourth operating unit 7 is deactivated.

Now the operator moves the holding system 1 to the operating table by means of the second operating unit 5 located in cavity 13 of the right lateral support 14. For this purpose, the operator actuates the operating button b21 and controls the holding system 1 by means of control element 12. A deflection of control element 12 in direction of control angle ϕ2 makes the platform 24 to move to the right or to the left. The deflection of the control element 12 in direction of control angle θ2 makes the platform 24 move forward or backward, while a deflection of control element 12 in direction of control angle ψ2 rotates the platform 24. When the holding system 1 has reached the operating table, the operator terminates the positioning of the holding system 1 in that he presses the additional operating button a2. The control element 12 is thereby deactivated.

The operator can control his position and/or orientation relative to the operating table by means of the third operating unit 6 by his own body movements. The adjustment axes v1 (height adjustment of operating chair 27), v2 (lateral adjustment of the operating chair 27) and v8 (traction means length of the upper body retaining device 30) are first in the holding mode H. Now the release device 8 for the adjustment axes v1, v2 is released by the operator by actuating the operating element b31 with the foot. The adjustment axes v1, v2 are now switched into the movement mode B, so that the operator can adjust the height of the operating chair 27, in that he loads or relieves the seat 28 of operating chair 27 using his legs.

During the adjustment of the height of seat 28, the height is detected by sensor unit s1 and transmitted to control device 10. As soon as the detected height remains at least substantially constant for a predetermined time I, the control unit 10 switches the adjustment axis v1 from the movement mode B into the holding mode H. By actuating the operating element b31, the control device 10 also switches the adjustment axis v2 from the holding mode H into the movement mode B. The adjustment axis comprises in this example two linear drives (only the one in front is depicted) that are respectively arranged laterally next to the operating chair 27. The two linear drives are attached to the operating chair 27 and the platform 24, so that a length change of the linear drives results in a pivot movement of the operating chair. The linear drives are operated in opposite sense, so that in case of a positive length change of one linear drive, the other linear drive carries out a negative length change.

During the adjustment of the lateral inclination of the operating chair 27, a force with which the upper body of the operator presses against the lateral supports 14, 16 is detected by sensor units s2 and s3 and is transmitted to the control device 10. If the operator presses his upper body against the right lateral support 14, the operating chair 27 pivots to the right. If the operator, however, presses his upper body against the left lateral support 16, the operating chair 27 pivots to the left. The control device 10 is configured to evaluate the force signals of sensor units s2, s3. As soon as the force signals have at least substantially the value 0 N for a predetermined time I, the control device 10 switches the adjustment axis v2 from the movement mode B into the holding mode H.

The operator can in addition adjust the inclination of his upper body, in that he operates the operating element b32 with the foot. By operating the operating element b32, the control device 10 switches the adjustment axis v8 of the upper body retaining device 30 from the holding mode H into the movement mode B. The operator is now able to vary the inclination of his upper body, whereby the length L_Z of traction means Z changes respectively. In the upper body retaining device 30 a respective sensor unit s4 is located, which is configured to detect the length L_Z of traction means Z. The sensor unit s4 transmits a respective sensor signal to the control unit 10 that is configured to evaluate the sensor signal. As soon as the control device 10 determines that a change of the length L_Z of traction means Z remains at least substantially constant, it switches the upper body retaining device 30 from the movement mode B into the holding mode H.

Additionally or alternatively, the control device 10 can be configured to switch the upper body retaining device 30 in a hover mode S, wherein the upper body retaining device 30 is configured to apply a traction force on the upper body of the operator via the traction means Z and the harness 25. With the traction force, movements of the operator are supported during which he inclines his upper body. For example, the control device 10 can be configured upon actuation of the release device 8 for a predetermined duration to activate the hover mode S of the upper body retaining device 30. For example, in this manner, the operator can keep the operating element b32 pressed for a predetermined duration, such as 0.5 s, 1 s or 1.5 s with his foot. If the operating element b32 comprises a light barrier, the operator can activate the hover mode S accordingly with his foot in that he keeps the foot for a predetermined duration in a release area of the light barrier. The operator can move freely and in accordance with the operation requirements in the hover mode S due to the permanently applied resetting force that is perceived by the operator as slight resistance without being required to repeatedly operate an operating unit for a position and/or orientation change. In this manner, the operator can move directly in the hover mode S without having to put down the instruments, wherein he is nevertheless relieved or supported to a certain degree. For example, the resetting force can be 60%, 70%, 80% or 90% of the force of gravity of the supported body part.

In this manner, the operator can control the support device 2 by his body movements without being required to put his instrument down. The operator can take a desired position and/or orientation in the movement mode B, which is set after termination of a short time in which the operator remains in this position. The simple adjustment of the position of the operator, which is maintained in the holding mode H by the support device 2, makes time-consuming repositionings of the support device 2 unnecessary. The operator can so concentrate on the operation and is less distracted by the operation of the holding system 1. The operation can thereby be carried out in a shorter time with a potentially higher quality.

All of the control commands that have been carried out by the operator can alternatively or additionally also be carried out by the assistant using the first operating unit 4.

The assistant can move the holding system 1 by means of the first operating unit 4 to the operating table and can reposition the holding system 1. For this purpose, the assistant operates the operating button b11 and controls the holding system 1 by means of the control element 11. A deflection of the control element 11 in direction of control angle ϕ1 makes the platform 24 move to the right or to the left. The deflection of the control element 11 in direction of the control angle θ1 makes the platform 24 move forward or backward, while a deflection of the control element 11 in direction of the control angle ψ2 rotates the platform 24.

In addition, the assistant can control the height and the lateral inclination of the operating chair 27 by deflection of the control element 11 in that he presses the operating element b12. The deflection of the control element 11 in direction of the control angle θ1 now controls the adjustment axis v1, while a deflection of the control element 11 in direction of control angle ϕ1 controls adjustment axis v2.

The assistant can also control the inclination of seat 28 in that he presses the operating element b13 and deflects control element 11 in direction of control angle θ1. Moreover, the assistant can adjust the lateral supports 14, 16 by actuation of operating element b14 and deflection of control element 11 in direction of control angle θ1. In addition, the assistant can wind traction means Z up or down by operating the operating element b15 and deflecting control element 11 in direction of control angle θ1 using the first operating unit 4.

When the holding system 1 has been brought in the desired position, the assistant can terminate the positioning of holding system 1 in that he presses the additional operating button a1. The control element 11 is thereby deactivated.

The holding system 1 according to the invention comprises a support device 2 having multiple adjustment axes v1, . . . , vn and an operating device 3. The support device 2 is configured to hold at least one body part of the operator supported by the support device 2 in a position and/or orientation. The operating device 3 is configured for control of the adjustment axes v1, . . . , vn and comprises at least one first operating unit 4 outside of the gripping range G of the operator and a second operating unit 5 arranged in the gripping range G of the operator, having at least partly coinciding functionality. Preferably, the operating device 3 comprises in addition a third operating unit 6 by means of which the operator can control the support device 2 by his body movements, as well as a fourth operating unit 7 arranged in the gripping range G of the operator. Thereby it is possible that the functionality defined by all adjustment axes of the support device 2 can be separated on the operating units 4, 5, 6, 7, whereby a particularly intuitive control of the adjustment axes v1, . . . , vn can be achieved for the operator.

### LIST OF REFERENCE SIGNS

1 holding system
2 support device
3 operating device
4 first operating unit
5 second operating unit
6 third operating unit
7 fourth operating unit
8 release device
9 sensor device
10 control device
11 control element of first operating unit
12 control element of second operating unit
13 cavity
14 lateral support
15 additional cavity
16 additional lateral support
17 forearm rest
18 additional forearm rest of additional lateral support
19 column support
20 first support arm
21 second support arm
22 foot rest
23 additional foot rest
24 platform
25 harness
26 back part of harness
27 operating chair
28 seat
29 additional support
30 upper body retaining device
31 control element of fourth operating unit
a1 additional operating element of first operating unit
a2 additional operating element of second operating unit
a4 additional control element of second operating unit
b11 . . . , b1n operating elements of first operating unit
b21 . . . , b2n operating elements of second operating unit
b41 . . . , b4n operating elements of fourth operating unit
B movement mode
F foot range of operator
G gripping range of operator
H holding mode
I predetermined time
P1 rest position of control element of first operating unit
P2 rest position of control element of second operating unit
S hover mode
s1 . . . , sn plurality of sensor units
v1 . . . , vn multiple adjustment axes
x1 first control axis of control element of first operating unit
y1 second control axis of control element of first operating unit
z1 third control axis of control element of first operating unit
ϕ1 first control angle of control element of first operating unit
θ1 second control angle of control element of first operating unit
ψ1 third control angle of control element of first operating unit
x2 first control axis of control element of second operating unit
y2 second control axis of control element of second operating unit
z2 third control axis of control element of second operating unit
ϕ2 first control angle of control element of second operating unit
θ2 second control angle of control element of second operating unit
ψ2 third control angle of control element of second operating unit
x4 first control axis of control element of fourth operating unit
y4 second control axis of control element of fourth operating unit
z4 third control axis of control element of fourth operating unit
ϕ4 first control angle of control element of fourth operating unit
θ4 second control angle of control element of fourth operating unit
ψ4 third control angle of control element of fourth operating unit
Z traction means

1. A holding system for an operator, comprising:
a support device comprising multiple adjustment axes which are configured to hold at least one body part of the operator supported by the support device in a position and/or orientation;
an operating device for control of the adjustment axes, which comprises at least a first operating unit arranged outside of a gripping range of the operator and a second operating unit arranged inside the gripping range of the operator, having at least partly coinciding functionality.

2. The holding system according to claim 1, wherein the adjustment axes that can be controlled by means of the second operating unit can also be controlled by the first operating unit.

3. The holding system according to claim 1, wherein the operating device comprises a third operating unit having a release device preferably arranged in a foot range of the operator, wherein the release device is configured to switch at least one of the adjustment axes of the support device from a holding mode into a movement mode and vice versa,
wherein the at least one body part of the operator supported by the support device can be moved in the movement mode and is held by the support device in the holding mode.

4. The holding system according to claim 3, wherein the third operating unit comprises a sensor device having a plurality of sensor units for detection of movements of the operator in adjustment direction of the adjustment axes and/or the support device in adjustment direction of at least one of the adjustment axes.

5. The holding system according to claim 4, wherein the sensor signals of the sensor device serve the third operating unit as control signals.

6. The holding system according to claim 3, wherein the operating device comprises a control device that is configured to switch the support device from the movement mode into the holding mode, if the at least one body part of the operator supported by the support device remains in a position for a predetermined time.

7. The holding system according to claim 3, wherein the adjustment axes that are controllable by the third operating unit are also controllable by the first operating unit.

8. The holding system according to claim 1, wherein the first operating unit comprises the following:
a control element, which is elastically supported in a rest position and manually movable along at least three control axes as well as
a plurality of operating elements that in case of actuation set different assignments for the control element between the control axes of the control element and the adjustment axes.

9. The holding system according to claim 8, wherein the first operating unit is configured to,
detect an amount and a direction of deflections of the control element out of the rest position carried out manually, as well as
assign a control signal to one of the adjustment axes of the support device depending on the amount and/or the direction of deflections of the control element out of the rest position carried out manually.

10. The holding system according to claim 1, wherein the second operating unit comprises the following:
a control element, which is elastically supported in a rest position and is manually movable along at least three control axes, as well as
a plurality of operating elements, which in case of actuation set different assignments for the control element between the control axes of the control element and the adjustment axes.

11. The holding system according to claim 10, wherein the second operating unit is configured to
detect an amount and a direction of a deflection of the control element out of the rest position carried out manually, as well as
assign a control signal to one of the adjustment axes of the support device depending on the amount and/or the direction of the deflection.

12. The holding system according to claim 6, wherein the control device is configured in case of at least substantially concurrent input at the first and second operating unit to control the adjustment axes based on the input at the second operating unit and to dismiss the input at the first operating unit.

13. The holding system according to claim 6, wherein the control device is configured to control the adjustment axes based on an input of the third operating unit and to dismiss inputs at the first operating unit if the third operating unit is switched into the movement mode.

14. The holding system according to claim 1, wherein the second operating unit is located in a cavity of a lateral support provided for the operator of the support device.

15. The holding system according to claim 1, wherein the first operating unit and the second operating unit comprise at least one additional operating element, which in case of actuation deactivates the a respective control element of the first operating unit and the second operating unit.

16. The holding system according to claim 2, wherein the operating device comprises a third operating unit having a release device preferably arranged in a foot range of the operator, wherein the release device is configured to switch at least one of the adjustment axes of the support device from a holding mode into a movement mode and vice versa,
wherein the at least one body part of the operator supported by the support device can be moved in the movement mode and is held by the support device in the holding mode.

17. The holding system according to claim 16, wherein the third operating unit comprises a sensor device having a plurality of sensor units for detection of movements of the operator in adjustment direction of the adjustment axes and/or the support device in adjustment direction of at least one of the adjustment axes.

18. The holding system according to claim 17, wherein sensor signals of the sensor device serve the third operating unit as control signals.

19. The holding system according to claim 18, wherein the operating device comprises a control device that is configured to switch the support device from the movement mode into the holding mode, if the at least one body part of the operator supported by the support device remains in a position for a predetermined time.

20. The holding system according to claim 19, wherein the adjustment axes that are controllable by the third operating unit are also controllable by the first operating unit.
